(19) United States
(12) Patent Application Publication
(10) Pub. No.: US 2014/0276117 A1
(43) Pub. Date: Sep. 18, 2014

(54) Intravascular Devices, Systems, and Methods

(71) Applicant: Volcano Corporation, San Diego, CA (US)

(72) Inventor: David H. Burkett, Temecula, CA (US)

(73) Assignee: Volcano Corporation, San Diego, CA (US)

(21) Appl. No.: 14/213,121

(22) Filed: Mar. 14, 2014

Related U.S. Application Data
(60) Provisional application No. 61/799,516, filed on Mar. 15, 2013.

(51) Int. Cl. A61B 5/00

(52) U.S. Cl. CPC A61B 5/6851; A61B 5/0084
USPC 600/479; 600/585; 29/428

(57) ABSTRACT

Intravascular devices, systems, and methods are disclosed. In some embodiments, the intravascular devices include at least one electronic, optical, or electro-optical component positioned within a distal portion of the device and one or more connectors positioned at a distal portion of the device. In some instances, a tubular member forming a main body of the intravascular device includes a variable, spiral cut along a distal section such that the tubular member has a variable stiffness along the length of its distal section. In some particular instances, the tubular member is a hypotube that has an increased flexibility as it extends distally as a result of the variable, spiral cut. In some instances, the pitch and/or width of the spiral cut is varied along the length of the distal section of the hypotube to increase flexibility. Methods of making and/or assembling such intravascular devices/systems are also provided.

### CROSS-REFERENCE TO RELATED APPLICATIONS

The present application claims priority to and the benefit of U.S. Provisional Patent Application No. 61/799,516, filed Mar. 15, 2013, which is hereby incorporated by reference in its entirety.

### TECHNICAL FIELD

The present disclosure relates to intravascular devices, systems, and methods. In some embodiments, the intravascular devices are guidewires that include one or more electronic, optical, or electro-optical components.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guidewires are utilized to measure the pressure within the blood vessel, visualize the inner lumen of the blood vessel, and/or otherwise obtain data related to the blood vessel. To date, guidewires containing pressure sensors, imaging elements, and/or other electronic, optical, or electro-optical components have suffered from reduced performance characteristics compared to standard guidewires that do not contain such components. For example, the handling performance of previous guidewires containing electronic components have been hampered, in some instances, by the limited space available for the core wire after accounting for the space needed for the conductors or communication lines of the electronic component(s), the stiffness of the rigid housing containing the electronic component(s), abrupt transitions in material properties between different components, and/or other limitations associated with providing the functionality of the electronic components in the limited space available within a guide wire.

Accordingly, there remains a need for improved intravascular devices, systems, and methods that include one or more electronic, optical, or electro-optical components.

### SUMMARY

Embodiments of the present disclosure are directed to intravascular devices, systems, and methods that include a flexible elongate member having a spiral cut to improve transitions in flexibility.

In one embodiment, a guide wire is provided. The guide wire comprises a main body including an elongate tubular member having a proximal section and a distal section, the distal section of the elongate tubular member having a variable, spiral cut; a flexible distal end coupled to the distal section of the elongate tubular member; at least one electronic, optical, or electro-optical components coupled to the flexible distal end; and at least one connector coupled to the proximal section of the elongated tubular member, the at least one connector in communication with the at least one electronic, optical, or electro-optical component.

In another embodiment, a method of assembling a guide wire is provided. The method includes providing an elongate tubular member having a proximal section and a distal section, the distal section of the elongate tubular member having a variable, spiral cut; coupling a flexible distal end to the distal section of the elongate tubular member, the flexible distal end including at least one electronic, optical, or electro-optical component; and coupling at least one connector to the proximal section of the elongated tubular member; and communicatively coupling the at least one connector to the at least one electronic, optical, or electro-optical component.

In another embodiment, a system is provided. The system includes a processing component in communication with a guide wire via at least one connector of the guide wire. The guide wire comprises a main body including an elongate tubular member having a proximal section and a distal section, the distal section of the elongate tubular member having a variable, spiral cut; a flexible distal end coupled to the distal section of the elongate tubular member; at least one electronic, optical, or electro-optical components coupled to the flexible distal end; and at least one connector coupled to the proximal section of the elongated tubular member, the at least one connector in communication with the at least one electronic, optical, or electro-optical component.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic side view of an intravascular device according to an embodiment of the present disclosure.

FIG. 2 is a diagrammatic schematic side view of a distal portion of the intravascular device of FIG. 1 according to an embodiment of the present disclosure.

FIG. 3 is a diagrammatic schematic side view of a distal portion of the intravascular device of FIG. 1 similar to that of FIG. 2, but illustrating another embodiment of the present disclosure.

FIG. 4 is a diagrammatic schematic side view of a distal portion of the intravascular device of FIG. 1 similar to that of FIGS. 2 and 3, but illustrating another embodiment of the present disclosure.

FIG. 5 is a diagrammatic schematic side view of a distal portion of the intravascular device of FIG. 1 similar to that of FIGS. 2-4, but illustrating another embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guidewires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a minor, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non- medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guidewire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

Referring now to FIG. 1, shown therein is a portion of an intravascular device 100 according to an embodiment of the present disclosure. In that regard, the intravascular device 100 includes a flexible elongate member 102 having a distal portion 104 adjacent a distal end 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a temperature sensor, an imaging element, an optical fiber, an ultrasound transducer, a reflector, a minor, a prism, an ablation element, an RF electrode, a conductor, and/or combinations thereof. The specific type of component or combination of components can be selected based on an intended use of the intravascular device. In some instances, the component 108 is positioned less than 10 cm, less than 5, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Further, in some embodiments the connector 110 provides both electrical and optical connections to both electrical conductor(s) and optical communication pathway(s) coupled to the component 108. In that regard, it should again be noted that component 108 is comprised of a plurality of elements in some instances. In some instances, the connector 110 is configured to provide a physical connection to another device, either directly or indirectly. In other instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. Generally, any number of electrical conductors, optical pathways, and/or combinations thereof can extend along the length of the flexible elongate member 102 between the connector 110 and the component 108. In some instances, between one and ten electrical conductors and/or optical pathways extend along the length of the flexible elongate member 102 between the connector 110 and the component 108. However, it is understood that a greater total number of communication pathways and/or the number of electrical conductors and/or optical pathways is provided in other embodiments. More specifically, the number of communication pathways and the number of electrical conductors and optical pathways extending along the length of the flexible elongate member 102 is determined by the desired functionality of the component 108 and the corresponding elements that define component 108 to provide such functionality.

In some implementations, the intravascular device 100 includes features (e.g., structures, components, designs, arrangements, communication pathways, connections, and/or other aspects) similar to those utilized in the intravascular devices of one or more of U.S. Provisional Patent Application No. 61/695,970, filed Aug. 31, 2012 and titled “MOUNTING STRUCTURES FOR COMPONENTS OF INTRAVASCULAR DEVICES,” U.S. Provisional Patent Application No. 61/665,697, filed on Jun. 28, 2012 and titled “INTRAVASCULAR DEVICES, SYSTEMS, AND METHODS,” and U.S. Pat. No. 7,967,762, filed Jan. 4, 2007 and titled “ULTRA MINIATURE PRESSURE SENSOR,” each of which is hereby incorporated by reference in its entirety. For example, in some implementations the proximal portion 102 of the intravascular device 100 includes features similar or identical to the proximal portion of an intravascular device disclosed in these earlier applications. Similarly, in some implementations the central portion of the intravascular device 100 (e.g., the portion associated with flexible elongate member 102) includes features similar or identical to the central portion of an intravascular device disclosed in these earlier applications. Likewise, in some implementations the distal portion 104 of the intravascular device 100 includes features similar or identical to the distal portion of an intravascular device disclosed in these earlier applications.

As shown in FIG. 1, a distal section 114 of the flexible elongate member 102 defines a transition between the flexible elongate member 102 and the distal portion 104 of the intravascular device. As discussed in greater detail below with respect to FIGS. 2-5, the distal section 114 of the flexible elongate member 102 includes a spiral cut that extends along the length of the intravascular device 100. In that regard, in some implementations the spiral cut serves to transition the stiffness of the intravascular device 100 between that of the flexible elongate member 102 and that of the distal portion 104. To that end, in some implementations the flexible elongate member 102 is stiffer, or less flexible, than the component(s) defining the distal portion 104 of the intravascular device 100. For example, in some instances the flexible elongate member 102 is a hypotube formed of a suitable medical grade material (e.g., Nitinol, Stainless Steel, etc.), while the distal portion 104 is defined by at least one coil and/or at least one coil-embedded polymer tube that is more flexible than the hypotube (See, for example, U.S. Provisional Patent Application No. 61/695,970, filed Aug. 31, 2012 and titled “MOUNTING STRUCTURES FOR COMPONENTS OF INTRAVASCULAR DEVICES,” U.S. Provisional Patent Application No. 61/665,697, filed on Jun. 28, 2012 and titled “INTRAVASCULAR DEVICES, SYSTEMS, AND METHODS,” and U.S. Pat. No. 7,967,762, filed Jan. 4, 2007 and titled “ULTRA MINIATURE PRESSURE SENSOR”). The present applicant has found that the relatively abrupt transition in flexibility between the flexible elongate member and the distal portion in traditional intravascular devices can result in poor handling characteristics of the intravascular device in some situations.

By introducing a spiral cut into the distal section 114 of the flexible elongate member 102, the intravascular devices of the present disclosure provide improved handling characteristics without sacrificing the functionality associated with the one or more electronic, optical, or electro-optical components. In general, the spiral cut of the distal section 114 serves to transition the flexibility of the intravascular device 100 between that of the flexible elongate member 102 and the distal portion 104. By transitioning the flexibility over the length of the distal section 114, an abrupt transition is avoided and a smoother, more continuous transition in flexibility is provided. To that end, in some instances the distal section 114 of the flexible elongate member 102 that includes the spiral cut has a length between about 1 cm and about 30 cm along the central longitudinal axis of the intravascular device 100, with some embodiments having a length between about 3 cm and about 8 cm. In some implementations, the distal section 114 is formed of a separate hypotube than the flexible elongate member 102. In some implementations the spiral cut varies along the length of the flexible elongate member 102 such that the flexibility of the flexible elongate member 102 increases as it extends distally along distal section 114. More specifically, the pitch and/or width of the spiral cut can be varied along the length of the distal section 114 of the flexible elongate member 102 to provide a desired transition in flexibility. In other embodiments, the spiral cut can have a substantially constant pitch and opening width along the length of the distal section 114 of the flexible elongate member 102.

Referring now to FIG. 2-5, shown therein are examples of variable, spiral cuts within a distal section of a flexible elongate member of an intravascular device according to various implementations of the present disclosure. Referring more specifically to FIG. 2, shown therein is an exemplary embodiment of the distal section 114 of the flexible elongate member 102 that includes a spiral cut 120 with a variable pitch. As shown, the spiral cut 120 extends from adjacent a proximal end 122 of the distal section 114 to adjacent the distal end 124 of the distal section 114. The pitch of the spiral cut 120 decreases as the spiral cut 120 extends distally from the proximal end 122 to the distal end 124. In the illustrated embodiment, the spiral cut 120 has a pitch 126 adjacent to the proximal end 122 that is larger than the pitch 128 adjacent the distal end 124. In some instances, the pitch 126 has a length along the longitudinal axis of the flexible elongate member 102 between about 0.1 mm and about 5.0 cm, with some particular embodiments being between about 5.0 mm and about 2.0 cm. In some instances, the pitch 128 has a length along the longitudinal axis of the flexible elongate member 102 between about 0.1 mm and about 5.0 cm, with some particular embodiments being between about 0.1 mm and about 2.0 cm. The transition in pitch of the spiral cut along the length of the flexible elongate member 102 can be a linear transition (as shown in FIG. 2), an exponential transition, a quadratic transition, other transition, and/or combinations thereof. In the embodiment of FIG. 2, the spiral cut 120 has a constant width along the length of the flexible elongate member 102. Accordingly, the opening defined in the sidewall of the flexible elongate member 102 by the spiral cut 102 has a constant profile, except for the change in pitch. In some instances, the width of the spiral cut 120 is between about 0.01 mm and about 5.0 mm.

Referring now to FIG. 3, shown therein is an exemplary embodiment of a distal section 130 of the flexible elongate member 102 that includes a spiral cut 132 with a variable width. As shown, the spiral cut 132 extends from adjacent the proximal end 122 of the distal section 130 to adjacent the distal end 124 of the distal section 130. The width of the spiral cut 132 increases as the spiral cut 132 extends distally from the proximal end 122 to the distal end 124. In the illustrated embodiment, the spiral cut 132 has a width 134 adjacent to the proximal end 122 that is smaller than the width 136 adjacent the distal end 124. In some instances, the width 134 has a length along the longitudinal axis of the flexible elongate member 102 between about 0.01 mm and about 5.0 mm, with some particular embodiments being between about 0.01 mm and about 1.0 mm. In some instances, the width 136 has a length along the longitudinal axis of the flexible elongate member 102 between about 0.01 mm and about 5.0 mm, with some particular embodiments being between about 0.1 mm and about 5.0 mm. The transition in width of the spiral cut along the length of the flexible elongate member 102 can be a linear transition (as shown in FIG. 3), an exponential transition, a quadratic transition, other transition, and/or combinations thereof. In the embodiment of FIG. 3, the spiral cut 132 has a constant spacing 138 between adjacent windings of the spiral cut along the length of the flexible elongate member 102. As a result of this constant spacing and the increase in width of the spiral cut 132, the pitch of the spiral cut 132 increases as the spiral cut 132 extends distally along the length of the flexible elongate member. In other embodiments, the spiral cut 132 with the variable width has a constant pitch along the length of the flexible elongate member.

Referring now to FIG. 4, shown therein is an exemplary embodiment of a distal section 140 of the flexible elongate member 102 that includes an intermittent spiral cut 142 with a variable width. In that regard, the spiral cut 142 is similar to spiral cut 132 described above in the context of FIG. 3 in many respects. As shown, the spiral cut 142 extends from adjacent the proximal end 122 of the distal section 140 to adjacent the distal end 124 of the distal section 140. The width of the spiral cut 142 increases as the spiral cut 142 extends distally from the proximal end 122 to the distal end 124 in the same manner as spiral cut 132. However, a distal section of the spiral cut 142 includes breaks or interruptions 144 where a section of the flexible elongate member 102 in the path of the spiral cut 132 is not removed. Accordingly, in the illustrated embodiment each of the interruptions 144 defines a bridge or connection across the spiral cut 142 that serves to maintain the tubular structure of the distal section 140 of the elongate tubular member. To that end, as the width of the spiral cut 142 is increased there is a corresponding increase in the likelihood that the bending of the distal section 140 as the intravascular device 100 is advanced through a tortuous path associated with an anatomical structure of interest will result in the distal section 140 kinking, which could cause problems with the functionality of the intravascular device as well as endanger the patient. Accordingly, in some instances the interruptions 144 in the spiral cut 142 are provided to prevent or at least reduce the likelihood that the distal section 140 of the flexible elongate member 140 will kink during use. The interruptions may be equally spaced (as shown in FIG. 4) or variably spaced along the length and/or circumference of the flexible elongate member 102. In some implementations, the interruptions are spaced between about ¾ of a revolution and 2 revolutions around the outer diameter of the flexible elongate member. Further, the size or length of the interruption along the length of the spiral cut 142 is between about 0.5 mm and about 1.0 mm in some instances.

Referring now to FIG. 5, shown therein is an exemplary embodiment of a distal section 150 of the flexible elongate member 102 that combines aspects of the distal sections 114, 120, 130, and 140 described above in the context of FIGS. 2-4. More specifically, the distal section includes an intermittent spiral cut 152 with a variable pitch and variable width. In that regard, the spiral cut 152 has a variable pitch that decreases as the spiral cut extends distally along the length of flexible elongate member, similar to spiral cut 120 of FIG. 2. Further, the spiral cut 152 has a variable width that increases as the spiral cut extends distally along the length of flexible elongate member, similar to spiral cut 132 of FIG. 3. Further still, the spiral cut 152 includes breaks or interruption 154 adjacent the distal end 124, similar to spiral cut 142 of FIG. 4. Those skilled in the art will recognize that a wide variety of spiral cuts having various combinations of constant pitch, variable pitch, constant width, variable width, no interruptions, interruptions, and/or combinations thereof may be utilized in accordance with the concepts of the present disclosure.

As shown in FIGS. 2-5, the distal end 124 of the distal section of the flexible elongate member 102 is positioned adjacent to the proximal end of the distal portion 104 of the intravascular device 100. In some instances, the component(s) of the distal portion 104 are coupled to the distal section of the flexible elongate member 102 using mechanical and/or chemical mechanisms. For example, in some implementations where the distal portion 104 is defined by at least one coil, the spiral cut of the distal section of the flexible elongate member 102 is sized and shaped to interface with the coil of the distal portion 104. For example, in some instances the coil threadingly engages the spiral cut of the flexible elongate member. Further, in some implementations where the distal portion 104 is defined by at least one coil-embedded polymer tube, the flexible elongate member 102 and the coil-embedded polymer tube are each secured to one or more core members of the intravascular device 100 using a suitable adhesive (e.g., epoxy, glue, etc.). Further, the flexible elongate member 102 and the coil-embedded polymer tube are secured to each other using a suitable adhesive (e.g., epoxy, glue, etc.) in some instances.

In some implementations, a flexible adhesive is applied inside the inner diameter of the spiral cut section of the flexible elongate member. In that regard, use of a flexible adhesive provides performance and safety benefits in some instances. For example, where the spiral cut flexible elongate member is used with a core or safety wire, the flexible adhesive attaches to the core/wire along the full length of the spiral cut section, minimizing torque loss. Further, the adhesive also anchors the polymer sleeve along the full length of the spiral cut. Also, the flexible adhesive will lock the conductive leads in place, minimizing the potential for wear of the insulation as the conductive leads are flexed against the spiral cut.

Further, in some implementations the use of a spiral cut hypotube also allows for the incorporation of a sensor mount (e.g., when using tubing with an outer diameter smaller than the desired maximum diameter of the guide wire) and/or a sensor housing (e.g., when using tubing with an outer diameter equal to or only slightly smaller than the desired maximum diameter of the guide wire). Utilizing such an approach can reduce the need for additional components and potential assembly concerns with those additional components. Use of the smaller OD tubing also eliminates the need for a joining component for the proximal hypotube connection.

In order to provide optimal guide wire functionality, the at least the spiral cut section of the flexible elongate member is covered with a polymer sleeve in some instances. The polymer sleeve provides several benefits, including: 1) acting as a substrate for adhesion of a lubricious hydrophilic coating; 2) minimizing potential pinching conditions in tighter tortuosity by covering the spiral cuts; and 3) serving as a wicking tube for application of the flexible adhesive (when used) by covering the spiral cuts.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A guide wire, comprising:
a main body including an elongate tubular member having a proximal section and a distal section, the distal section of the elongate tubular member having a variable, spiral cut;
a flexible distal end coupled to the distal section of the elongate tubular member;
at least one electronic, optical, or electro-optical components coupled to the flexible distal end; and
at least one connector coupled to the proximal section of the elongated tubular member, the at least one connector in communication with the at least one electronic, optical, or electro-optical component.

2. The guide wire of claim 1, wherein a pitch of the spiral cut varies along a length of the elongate tubular member.

3. The guide wire of claim 2, wherein the pitch of the spiral cut decreases as the spiral cut extends distally towards the flexible distal end.

4. The guide wire of claim 3, wherein the flexible distal end includes a coil that is at least partially received within a part of the spiral cut.

5. The guide wire of claim 4, wherein the coil threadingly engages the part of the spiral cut.

6. The guide wire of claim 2, wherein a width of the spiral cut is constant along the length of the tubular member.

7. The guide wire of claim 2, wherein a width of the spiral cut varies along the length of the tubular member.

8. The guide wire of claim 7, wherein the width of the spiral cut increases as the spiral cut extends distally towards the flexible distal end.

9. The guide wire of claim 1, wherein a width of the spiral cut varies along the length of the tubular member.

10. The guide wire of claim 9, wherein the width of the spiral cut increases as the spiral cut extends distally towards the flexible distal end.

11. A method of assembling a guide wire, comprising:
providing an elongate tubular member having a proximal section and a distal section, the distal section of the elongate tubular member having a variable, spiral cut;
coupling a flexible distal end to the distal section of the elongate tubular member, the flexible distal end including at least one electronic, optical, or electro-optical component; and
coupling at least one connector to the proximal section of the elongated tubular member; and
communicatively coupling the at least one connector to the at least one electronic, optical, or electro-optical component.

12. The method of claim 11, wherein a pitch of the spiral cut varies along a length of the elongate tubular member.

13. The method of claim 12, wherein the pitch of the spiral cut decreases as the spiral cut extends distally towards the flexible distal end.

14. The method of claim 13, wherein the flexible distal end includes a coil that is at least partially received within a part of the spiral cut.

15. The method of claim 14, wherein the coil threadingly engages the part of the spiral cut.

16. The method of claim 12, wherein a width of the spiral cut is constant along the length of the tubular member.

17. The method of claim 12, wherein a width of the spiral cut varies along the length of the tubular member.

18. The method of claim 17, wherein the width of the spiral cut increases as the spiral cut extends distally towards the flexible distal end.

19. The method of claim 11, wherein a width of the spiral cut varies along the length of the tubular member.

20. The method of claim 19, wherein the width of the spiral cut increases as the spiral cut extends distally towards the flexible distal end.
